(19) United States
(12) Patent Application Publication
(10) Pub. No.: US 2011/0015762 A1
(43) Pub. Date: Jan. 20, 2011

(54) JOINTS FOR PROSTHETIC, ORTHOTIC AND/OR ROBOTIC DEVICES

(75) Inventor: Jerome R. Rifkin, Louisville, CO (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: Tensegrity Prosthetics Inc., Louisville, CO (US)

(21) Appl. No.: 12/630,934

(22) Filed: Dec. 4, 2009

Related U.S. Application Data
(60) Provisional application No. 61/225,439, filed on Jul. 14, 2009.

(51) Int. Cl. A61F 2/66

(52) U.S. Cl. 623/53

(57) ABSTRACT

An artificial foot device may include a talus body, a core operatively coupled with the talus body by a first joint, and a toe operatively coupled with the core by a second joint. The first joint may provide for constrained relative movement between the talus body and the core. The second joint may provide for constrained relative movement between the core and the toe. Constrained relative movement between the talus body and the core may substantially correspond to a coordinated movement of a first natural joint and a second natural joint during ambulation of a natural human foot.

### REFERENCE TO RELATED APPLICATIONS

This application is related to pending U.S. patent application Ser. No. 12/464,747, filed May 12, 2009, which claims the benefit of priority of provisional U.S. Patent Application No. 61/127,482, filed May 13, 2008, the entirety of which are incorporated herein by reference.

This application is also related to pending U.S. patent application Ser. No. 11/080,972, filed Mar. 16, 2005, which claims the benefit of priority of provisional U.S. Patent Application No. 60/553,619, filed Mar. 16, 2004, the entirety of which are incorporated herein by reference.

This application claims the benefit of priority of provisional U.S. Patent Application No. 61/225,439, filed Jul. 14, 2009, the entirety of which is incorporated herein by reference.

### TECHNICAL FIELD

This application relates generally to artificial foot devices, such as a prosthetic, orthotic or robotic foot that simulates the coordinated motions of the natural human foot, particularly in walking gait. More particularly, embodiments of this application relate to a prosthetic, orthotic or robotic foot including three segments connected by two joints: one joint analogous to the human first metatarsophalangeal joint, and the other joint analogous to the human subtalar joint.

### BACKGROUND

People who lose a leg today may be in a bad situation. Some days, a simple staircase may seem like an insurmountable challenge. Walking up a grassy slope is too difficult to attempt, because multiple falls may be inevitable. War, accidents and disease keep this disadvantaged population growing. Prosthetics, or synthetic replacements for missing anatomical structures, hold the promise of restoring some of this lost function and improving quality of life.

Just trying to regain functional mobility, amputees spend an average of $8,000 on below-knee (BK) prosthetic legs that last three to five years. Rather than spend this money on costly, non-repairable devices, one hundred and twenty thousand American amputees have chosen crutches or wheelchairs, and they won't walk again.

Just as the speed of a vehicle is maintained through regular energetic pushes received from pistons firing in the engine, normal human gait relies on well-timed pushes from the anatomy of the foot, during the toe-off portion of the walking cycle. Providing a suitable timing of toe-off, while providing a stable, level base—a preferable innovation addressed in this application—is lacking from existing feet prostheses and may be relevant for natural and comfortable ambulation.

The human gait is in reality a very complex process that at a basic level may be described as a series of repeating operations carried out by a single leg: 1) initial heel strike, 2) double support as both feet contact the ground, 3) stance phase as one leg supports the entire body weight, 4) pre-swing or heel-rise as the heel rises from the ground, 5) toe-off as the moment that the toes lose contact with the ground, and finally 6) swing phase, where the leg, acting as a pendulum, comes forward in preparation to repeat the process. In a two legged description of pre-swing, the heel of the contralateral leg strikes the ground at the exact moment that the ipsilateral heel rises. This is called double stance phase, and may be relevant to understanding the innovations presented in this application. Coordinated movement between the legs and the overall balance and trajectory of the body dynamic may be also relevant to successful ambulation.

Currently, there are two dominant paradigms of prosthetic foot design: post-like, conventional feet (CF) and leaf-spring-like, energy storing feet (ESF). Both of these designs change shape under loading, in an attempt to mimic the human foot. The classic CF foot, also known as the Solid Ankle Cushioned Heel (SACH), foot may provide a stable base for support, and is functionally unchanged since its conception in the 1960's. Introduced in the 1980's, carbon-fiber, leaf-spring ESF designs allow amputees to run by mimicking the ankle plantar flexors, returning energy to their stride. Para-lympic records rivaling their Olympic counterparts show that the ESF paradigm works very well for running, but studies have failed to show that these benefits extend to walking. 40% of transtibial amputees do not use prostheses and 78% of transfemoral amputees forego this intervention. Thus, over 120,000 amputees do not use prosthetic legs, preferring wheelchairs or crutches, never walking again. Studies of amputee psychosocial adjustment have linked positive emotional coping and higher levels of physical independence.

Depending on the type of foot used, CF or ESF, and the specific manufacturer, there have been subtle but significant differences in parameters such as stride length, symmetry of stride, and timing of the various phases of gait. For either foot type, stride length is shorter for strides where the prosthesis is the supporting limb, gait symmetry is markedly decreased, and the timing of the phases of gait may be disrupted. Most notably, there is a shortened stance phase on the prosthesis, a late toe off, and a longer swing phase on the affected side as well. Studies also describe an early incidence of low back and patellar-femoral osteoarthritis in unilateral amputees. The literature clearly shows that current prostheses fail to walk like an intact limb. In fact, clinical prostheticists have expressed the opinion that some “middle ground” between the unsophisticated CF feet and the highly athletic ESF feet is needed. Embodiments of the invention outlined here may be just that middle ground.

To lay the foundation for the rest of this submission, a few questions may be asked. Precisely how may an intact limb walk? And what is the role of the foot in this process? To address the first question, this application may present two different types of engineering control systems, and may provide illustrative examples. To address the second question, more studies may be presented, furthering the discussion, showing results of highly detailed, instrumented gait studies of the foot. Comparisons between the functional movements of the human foot, and the functional movements of current prostheses may follow. The improvements embodied in embodiments of the proposed device may address many of the shortcomings seen in the current technology.

With all of the myriad muscles and bones in human hips, legs, and feet, there is no “right” answer for how to propel one's self across a room or up a slope; however, there may be more optimal solutions, for example, ones that may be less abusive to the anatomy and/or ones with more optimal energetic efficiency. Early incidence of osteoarthritis, a degenerative joint change, is one indicator of a suboptimal movement strategy.

There may be many ways to walk, and data shows that people don't walk in exactly the same way with each stride. The hips may work harder on some strides than others; sometimes the lower leg may contribute varying amounts torque to the stride. Walking from one's hips may be described as a “top-down” control mechanism, where forces from the proximal leg may dictate the position and accelerations of the distal structures. This mechanism is very clearly illustrated in above knee (AK) amputees. Until recent, expensive innovation of computer controlled knees, AK amputees who wanted to walk faster than the return rate of their knee spring had to use a “hip snap,” flinging their prosthesis out quickly with their hip flexors, and then quickly contracting their hip extensors to snap the prosthetic knee straight in time for heel-strike. Thus, the anatomic ranges of motion guided the position of the prosthetic anatomy, but the timing the movement was controlled by the hip, in a “top-down” fashion.

A “top-down” control mechanism may also be seen in studies of trans-tibial amputees. The iEMG data of one study showed a greater use of the biceps femoris (BF) as compared to the antagonistic vastus medialus (VM) in the amputated limb, as opposed to the normal limb. The mean ratios of BFNM activity during the first half of stance phase was 3.8 in the amputated limb and 2.0 in the sound leg, with a P value of less than 0.042. Furthering elaboration on the “top-down” nature of this control system, an exceptionally statistically rigorous study from 2002 revealed some interesting trends in the flexor/extensor ratios for the knees of unilateral, trans-tibial amputees, as compared to normal volunteers. Though the amputees were much weaker than the normal control group, this study showed that there was no significant difference between the knee flexor/extensor ratios for peak bending moment, total work, or maximum power comparing either leg of the amputees and either leg of the non-amputees. Of course, the BF and VM may be also knee flexors and extensors, but not during the relevant time-span cited by the first study, early stance phase. Considering these studies together, one may conclude that trans-tibial amputees use the hip of the amputated leg more than the hip of their sound leg, and that they use their knee flexors and extensors normally. Clearly, the control mechanism being employed in a trans-tibially amputated limb is “top-down.”

The overuse of a particular muscle must result in overuse of the surrounding and supporting muscles. For example over loading a hip muscle causes the hip stabilizers to be over-recruited. If multifidus and transversus abdominus, the deepest pelvic stabilizers, may be overwhelmed, the larger quadratus lomborum (OL) and erector spinae (ES) muscles that may be normally used for motion may be recruited to help it. When the QL and ES are used as stabilizers, the agonists may also be recruited as stabilizers, just as transverses abdominus is recruited along with multifidus. When the QL and ES become a routine part of the stabilizing muscle pattern, they become tonic and rigid. Thus, putting a great deal of compression on the spine. This is a well-known pattern of muscle use and, if allowed to progress unchecked, may eventually result in degenerative joint changes in the lower spine.

Walking from the foot, as opposed to the hip, may be modeled as a “bottom-up” control scheme, where the distal anatomy directs the position of the proximal anatomy. The coordination of the metatarsophalangeal joint (MTP) of the great toe and the subtalar joint may create a dynamic in gait where the proximal foot and tibia subtly change angular position. This angular change may be the start of building momentum for toe off. In context of the gait cycle, starting from single stance phase, as the tibial shaft moves past perpendicular and over the foot, the subtalar joint may be eccentrically loaded. This may be seen as a “flatter” transverse arch. This subtle motion may progress with the tibial shaft advancement, with a maximum angular change of 10 degrees. In double stance phase, much of this weight may be off-loaded to the other leg, but the transverse arch may not yet spring back into shape. In fact, this new conformation may be maintained until just after heel rise. When the heel leaves the ground, passing the remaining force loading to the ball of the great toe, the MTP of the great toe may be forced into extension. This motion may pull on the plantar aponeurosis, which in turn may pull on the calmayeus and the Achilles tendon. This action may loft the transverse arch back to its stance phase conformation, subtly altering the position of the ankle and the tibia, and thus may change position of the knee and hip.

The relevant anatomy for this coordination of the first MTP and subtalar joints is well documented. The plantar aponeurosis spans both joints, as may the tendon of the flexor hallucis longus. Different research references attribute this coordination to each of these sources. The action of arching the subtalar joint by forcibly extending the first MTP has been described as the Windlass mechanism, and this passive, non-muscular change may be a function of timing and anatomic length. This timing may be influenced by the peronii, the tibialis anterior, and the intrinsic foot muscles. Of course, a passive prosthesis may not duplicate the action of these muscles, but it may mimic the action of the plantar aponeurosis. Due to the quasi-psuedoviscoelastic nature of the plantar aponeurosis and the surrounding musculature, this quick lofting of the plantar arch may be an energy storage mechanism. The energy may then be released, a moment later, on toe off. As seen in the temporal gait asymmetry of amputees, most notably in late stance and swing phases, studies have shown conclusively that this action is not accomplished in either CF or ESF designs.

These two distinct “ways of walking” represent extremes, and, as human nature dictates, we all walk with a varying degree of each mechanism. Amputees must rely exclusively on the strategy of top-down control, resulting in an overcompensation of the remaining anatomy which in turn may cause early degenerative changes. What is needed is a prosthesis that accurately imitates the relevant biomechanics of the natural foot, allowing for the contributions of the more efficient “bottom-up” gait style.

There is a definite coordination between the joints of the foot. The angular relationship shown between the forefoot and hallux may be the angular position of the first MTP. The angular motion between the forefoot and hindfoot may reflect the motion of the subtalar joint. A few studies have explored the detailed biomechanics of the foot using this powerful analytical technique, but they did not combine the detailed foot analysis with the protocol for the rest of the body. Thus, no quantified joint powers were generated. Experts may also be aware of the subtle, but highly significant errors in instrumented gait analysis of ESF prosthesis gait. Failure to accurately model the center of curvature of the leaf spring foot, for the purpose of reverse engineering the joint torques, may be the documented source of this error. The standard seven segment lower body model, used to reverse engineer joint torques, may use a rigid single segment foot. This simplified model may leave out both the first MTP and the subtalar joints, masking the relevant contribution of the Windlass mechanism, a subtle “bottom-up” contributor of gait mechanics. Theoretically, a nine segment lower body model, as seen in computer simulations, may show sensitivity to changes in spring stiffness of the MTP joint at push off, but still may exclude the subtalar joint or any coordination of the two.

The movement of the subtalar joint and first MTP during stance phase and toe-off, as described above, may correlate to a relatively new area of prosthetics research. Roll-over shape may be defined as the geometry a foot/ankle complex takes during the single limb stance phase of walking. As the center of weight may pass over the long axis of the prosthetic foot, it may bend according to its stiffness. The shape described by this bending may be the rollover-shape, and it may be defined in general terms as a rigid rocker model of the foot/ankle complex. A three dimensional rollover shape may be called a rollover surface, and a two dimensional shape may be called a rollover profile.

Studies of various prosthetic feet with the rollover profile methodology have shown that the “effective foot length” during walking is surprisingly short in many cases. For example, a size 28 cm SACH foot may display a functional length of less than 20 cm. The length of the rollover profile is significant for many gait parameters, and recent studies show that it may be relevant to how much oxygen is consumed during gait.

Considering the rollover profile length, along with the recent research into oxygen consumption dynamics, points toward a discrepancy that may be more significant than previously thought. In fact, the energy used in walking may be proportional to the fourth power of the step length. Since the stride length may be equal to the functional foot length plus the distance covered by swing phase, feet with shorter rollover profiles may deliver shorter stride lengths. The average step length is about 0.75 meters, and the difference in rollover profile between a SACH foot and a flex-foot is about 6 centimeters. Considering the relationship described above, one would anticipate a large energy savings by using the longer flex-foot, because the step length is almost 10% greater for the ESF versus over the CF. Surprisingly, this energy savings is not seen in any ESF models with longer rollover profiles. In fact, research shows a small energy savings, on the order of 3%, and some of the research subjects in that study found that some ESF feet were more tiring to use than some CF feet. This correlates well with the experience of clinical prosthetists, who describe that their patients often work against their ESF feet, because their return of power is not biomechanically accurate. Indeed, studies of prostheses show that a very small component of this energy return is in the antero-posterior direction, unlike the natural human limb.

### SUMMARY

There is a need for improved artificial foot devices. In particular, there is a need for artificial foot devices that more accurately simulate the motion and or function of the human foot during walking.

In one embodiment, an artificial foot device may be provided. The artificial foot device may include: a talus body, a core operatively coupled with the talus body by a first joint that provides for constrained relative movement between the talus body and the core; and a toe operatively coupled with the core by a second joint that provides for constrained relative movement between the core and the toe.

In some embodiments, the first joint may permit limited relative rotation of the talus body and the core about a first lateral axis, with the talus body, the core and the toe defining a longitudinal direction of the artificial foot. Alternatively or additionally, the first joint may permit limited relative rotation of the talus body and the core about a first longitudinal axis. Alternatively or additionally, the first joint may permit limited relative rotation of the talus body and the core about a substantially vertical axis.

In some embodiments, the second joint may permit limited relative rotation of the core and the toe about a first lateral axis. Alternatively or additionally, the second joint may permit limited relative rotation of the core and the toe about a first longitudinal axis. Alternatively or additionally, the second joint may permit limited relative rotation of the core and the toe about a substantially vertical axis. Alternatively or additionally, the second joint may permit limited relative lateral movement between the core and the toe.

In some embodiments, the first joint may include means for constraining relative movement between the talus body and the core other than about a lateral axis. In such embodiments, the means for constraining relative movement between the talus body and the core other than about a lateral axis may be configured to constrain relative movement between the talus body and the core about a longitudinal axis and/or about a substantially vertical axis.

In some embodiments, the second joint may include means for constraining relative movement between the core and the toe other than about a lateral axis. In such embodiments, the means for constraining relative movement between the core and the toe other than about a lateral axis may be configured to constrain relative movement between the core and the toe about a longitudinal axis and/or about a substantially vertical axis.

In some embodiments, the constrained relative movement between the talus body and the core may substantially correspond to a coordinated movement of a first natural joint and a second natural joint during ambulation of a natural human foot. In such embodiments, the constrained relative movement between the core and the toe may substantially correspond to a coordinated movement of a third natural joint, different from the first and second natural joints, during ambulation of a natural human foot.

In some embodiments, the artificial foot may include a coordination member operatively coupled with the talus body and the toe. In such embodiments, the coordination member may be configured to store and release energy during a walking movement of the artificial foot.

In some embodiments, the artificial foot may include a coordination member operatively coupled with the talus body and the core. In such embodiments, the coordination member may be configured to store and release energy during a walking movement of the artificial foot.

In some embodiments, the artificial foot may include at least one member operatively coupled with the core and the toe. In such embodiments, the at least one member may be configured to store and release energy during a walking movement of the artificial foot.

In some embodiments, a method of providing motion in an artificial foot device may be provided. The method may include coordinated movements of three or more structural members operatively coupled by two or more joints. The coordinated movements may be constrained by the two or more joints and/or interactions between the three or more structural members.

In some embodiments, a method of providing motion in an artificial foot device may include constrained relative movement between a first structural member and a second structural member. Such constrained movement may substantially correspond to a coordinated movement of a first natural joint and a second natural joint during ambulation of a natural human foot.

In such embodiments, the method may include constrained relative movement between the second structural member and a third structural member. Such constrained movement may substantially correspond to a coordinated movement of a third natural joint, different from the first and second natural joints, during ambulation of a natural human foot.

### BRIEF DESCRIPTION OF THE DRAWINGS

These and other features, aspects and advantages will be more fully understood when considered with respect to the following detailed description, appended claims and accompanying drawings, wherein:

FIGS. 1-7 illustrate an artificial foot including a talus body, a core and a toe according to an embodiment of the invention.

FIG. 8 illustrates an exploded view of the artificial foot illustrated in FIGS. 1-7.

FIGS. 9-13 illustrate views of the talus body of the artificial foot illustrated in FIGS. 1-8.

FIGS. 14-19 illustrate views of the core of the artificial foot illustrated in FIGS. 1-8.

FIGS. 20-26 illustrate views of the toe of the artificial foot illustrated in FIGS. 1-8.

FIGS. 27-30 illustrate views of the encasement of the core spring carrier assembly of the artificial foot illustrated in FIGS. 1-8.

FIGS. 31-35 illustrate views of the encasement of the toe spring carrier assembly of the artificial foot illustrated in FIGS. 1-8.

FIGS. 36-38 illustrate views of one of the links for coupling the core and toe of the artificial foot illustrated in FIGS. 1-8.

### DETAILED DESCRIPTION

Various details are described below, with reference to illustrative embodiments. It will be apparent that the invention may be embodied in a wide variety of forms, some of which may be quite different from those of the disclosed embodiments. Consequently, the specific structural and/or functional details disclosed herein are merely representative and do not limit the scope of the invention.

For example, based on the teachings herein it should be understood that the various structural and/or functional details disclosed herein may be incorporated in an embodiment independently of any other structural and/or functional details. Thus, an apparatus may be implemented and/or a method practiced using any number of the structural and/or functional details set forth in the disclosed embodiments. Also, an apparatus may be implemented and/or a method practiced using other structural and/or functional details in addition to or other than the structural and/or functional details set forth in the disclosed embodiments.

Embodiments of the device proposed in this application may be based on the tensegrity design idea. Tensile-integrity, shortened to “tensegrity,” may refer to a special type of structure comprising continuous tensile members, such as cables, acting upon discontinuous compressive members (e.g., spars). Tensegrity structures may rely upon the tensile strength and flexibility properties of wire rope to bear physical loads placed upon them. Major innovations in steel wire rope technology, driven by increasing performance demands in the automotive and aerospace sectors, now permit the construction of light weight joints that may be stronger in many cases than traditional engineered “beam and bearing” structures. Also, a solid link, such as a rod, that is not free to rotate to an orientation of pure tension may be employed.

As used herein, “tensegrity” may refer to the characteristic of having two or more discontinuous members dispersed in a network of one or more continuous tension members.

As used herein, “tensegrity joint” may refer to a joint having a tensegrity structure. In a tensegrity joint, the two or more discontinuous members may be incompletely constrained by the network of the one or more continuous tension members in which they may be dispersed, whereby the members may be able to move relative to each other. The movement may be at one or more centers of motion, and optionally around a primary axis at each center. Optionally, the primary axis may be virtual and not coaxial with an actual tension member. As described herein, in addition to the constrained or limited range of relative motion of the discontinuous members of the joint provided by the tension member(s), contact between the discontinuous members may also constrain or limit the relative motion.

As referred herein, dorsiflexion may be defined as motion in the direction of the top of the foot (e.g., dorsal surface), and plantarflexion may be defined as motion in the direction of the bottom of the foot (e.g., plantar surface).

In a tensegrity joint, the discontinuous members may be rigid, and the number, length, diameter, geometric organization, and flexibility characteristics of the tension members may determine the range of motion of the discontinuous members. Tension members may constrain and/or stabilize the discontinuous members.

Further, a tensegrity approach may be employed to couple movements of multiple joints. For example, the movement or motion of a first joint may be coupled to movement/motion of a second joint via one or more tension members, such as a coupling cable. The coupling cable(s) may connect two discontinuous members that may not be connected by a single joint, but indirectly connected by two or more joints.

Each joint in the animal body may have its own specific geometry. Joint(s) in embodiments of the invention may be designed to have similar characteristics of natural joints. Alternatively, super joints may be designed for prosthetics, orthotics, and robotics that do not interfere with the functioning of the remaining joints of the body or robot.

The materials of the discontinuous and tension members may be selected to maintain structural integrity considering the use of the device and the user of the device. Devices that must withstand greater forces may be made from stronger materials.

As used herein, “rope” may refer to an element capable of functioning as a tension member in a tensegrity joint, such as cables having a diameter less than about ¼ inch, for example. An effective rope element may comprise two or more thinner ropes, including a thin rope making multiple passes and having a larger effective diameter than the thin rope alone. Generally, these ropes may not be elastic.

As used herein, “rod” may refer to an element capable of functioning as a tension member in a tensegrity joint as well. It should be understood that the rod described in the particular embodiments herein is only an example.

Orthotics may augment body parts. Prosthetics may replace body parts. Robotics may function similarly to body parts, but may not require direct connection to a body in order to be functional.

Limit ropes and stabilization ropes may be tension members in tensegrity joints that limit the ranges of motion of the joint. Optionally, the tensegrity joint may have a primary axis of motion. A primary axis of motion may be the least constrained axis of all the other axes of motion of the joint. Optionally, one tension member may be coaxial with the primary axis of motion of the joint. Alternatively, the primary axis of motion may not be coaxial with a tension member, and the primary axis may then be said to be virtual.

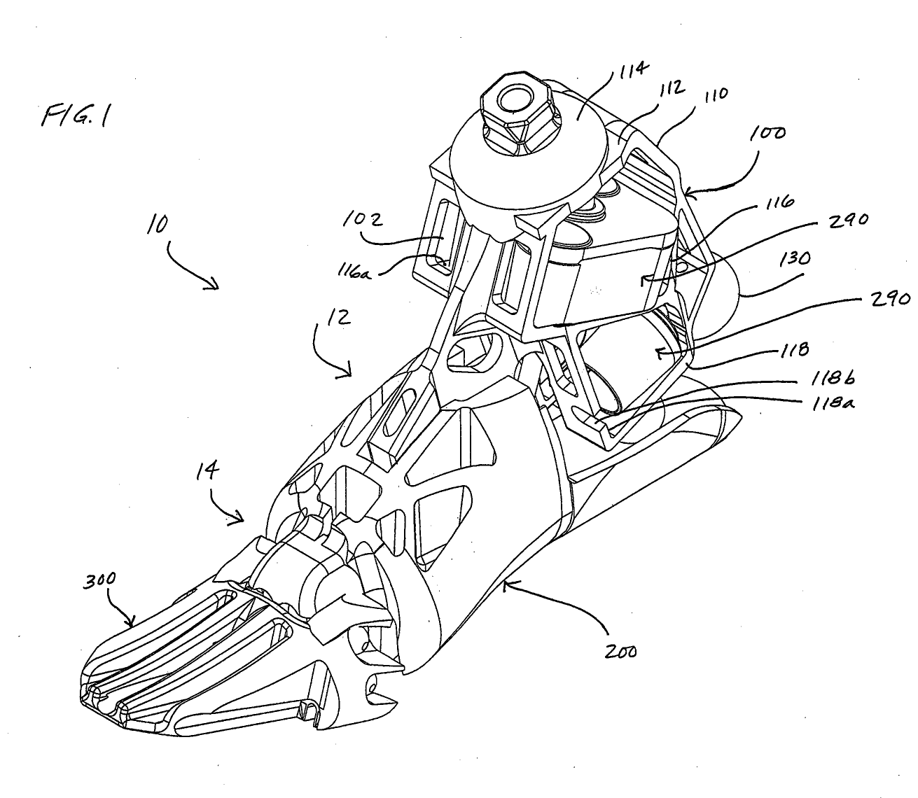

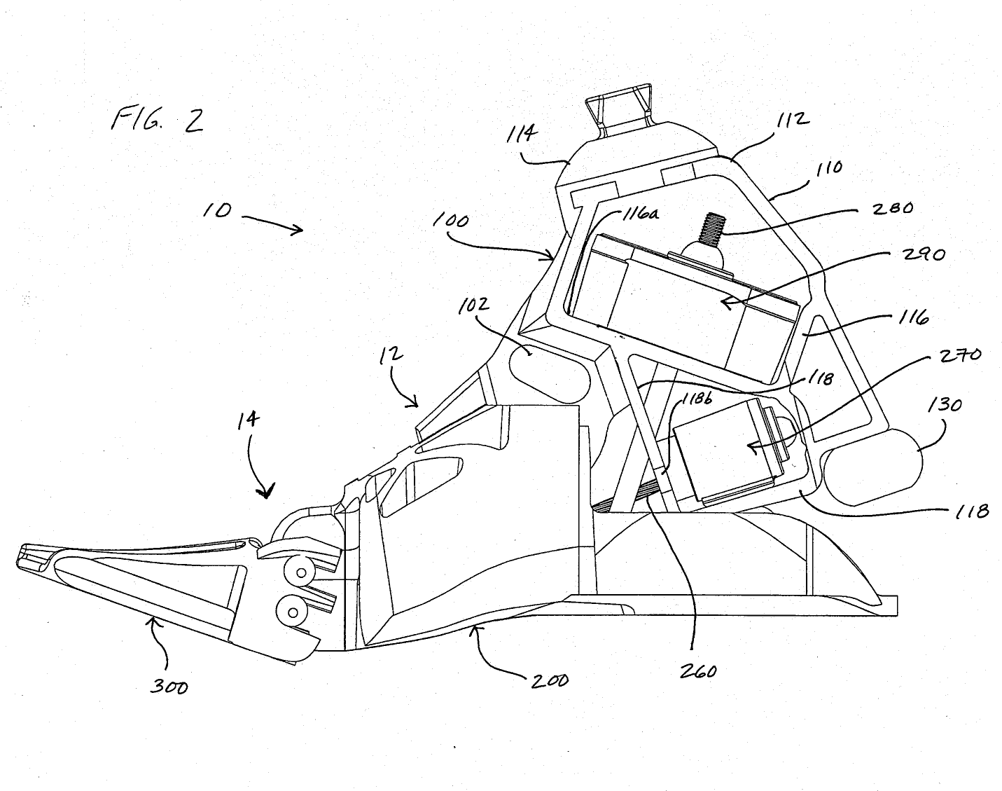

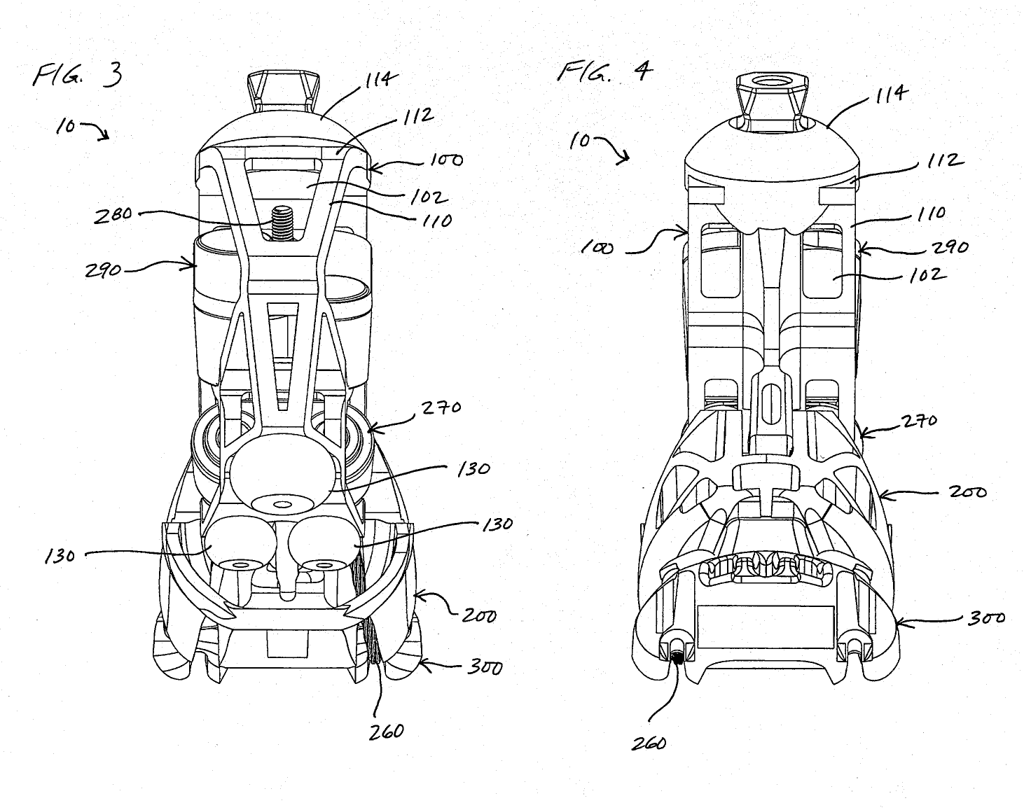

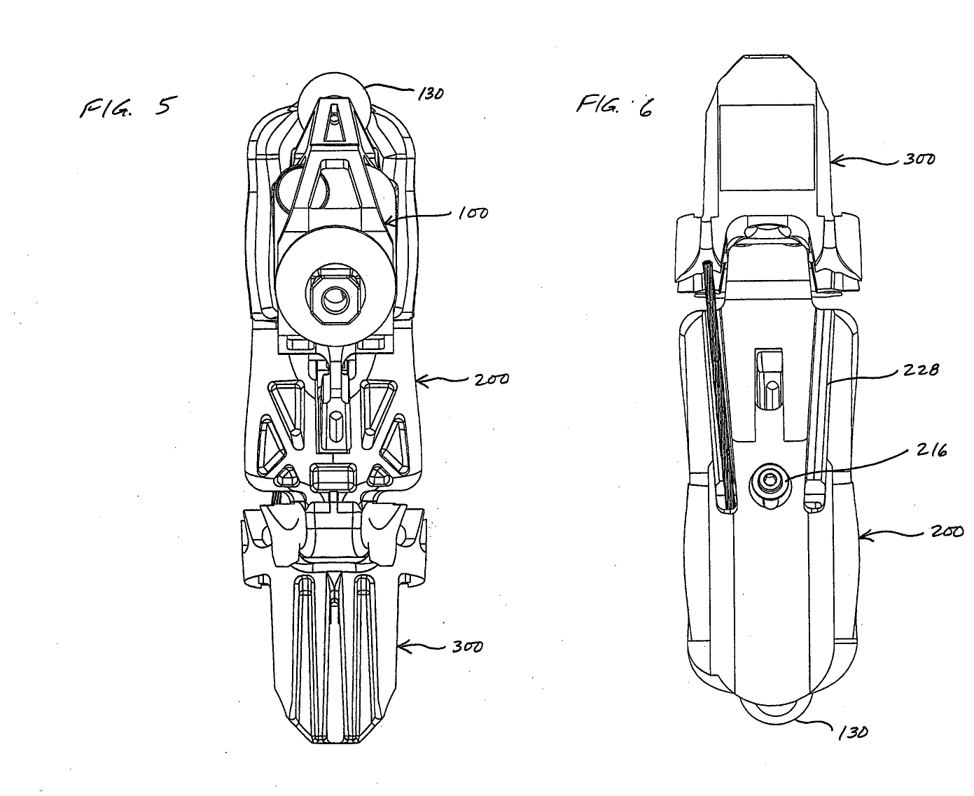

FIGS. 1-8 illustrate an artificial foot 10 including a talus body 100, a core 200 and a toe 300 according to an embodiment of the invention. FIG. 1 depicts a front perspective view of the artificial foot 10. FIG. 2 depicts a left side view of the artificial foot 10, the right side view of the artificial foot 10 being identical in this embodiment. FIG. 3 depicts a rear view of the artificial foot 10. FIG. 4 depicts a front view of the artificial foot 10. FIG. 5 depicts a top view of the artificial foot 10. FIG. 6 depicts a bottom view of the artificial foot 10. FIG. 7 is an exploded view of the artificial foot 10. FIG. 8 is a complete assembly of the artificial foot 10.

The talus body 100 may be operatively coupled with the core 200 via a first joint 12. The core 200 may be operatively coupled with the toe 300 via a second joint 14. As described herein, it should be understood that the first and second joints 12, 14 include one or more tension/limit members, such as ropes, some of which are not shown in these FIGS. for simplicity. As discussed above, the tension/limit member(s) may constrain movement of the talus body 100, the core 200 and the toe 300.

Although not shown in FIGS. 1-7, it should be understood that a pylon may be coupled to the talus body 100, for example, when the artificial foot 10 is configured to serve as a prosthesis. As discussed herein, the artificial foot 10 may provide a more natural walking motion for the user because of the joints 12, 14 operatively coupling the talus body 100, the core 200 and the toe 300. Details of specific embodiments of the talus body 100, the core 200 and the toe 300 are discussed below.

The talus body 100 may be described as including a frame structure 110 and a front section 120, extending generally in a direction of the toe 300 when the artificial foot 10 is assembled. The talus body 100 may be made of a suitably strong, structural material, such as stainless steel. The talus body 100 may be machined, for example, with various cutouts 102 for weight savings. Alternatively, the talus body 100 may be cast, for example, using a suitable steel.

An upper portion 112 of the frame structure 110 of the talus body 100 may include a coupling or engagement means 114 configured to couple/engage with a pylon, for example, as in the case of the artificial foot being implemented as a prosthesis. The coupling/engagement means 114 may be in the form of a well-known “pyramid connector” and may be formed as an integral part of the frame structure 110, as appropriate or desired, for example, whether by machining or casting.

A mid-portion 116 of the frame structure 110 may provide a substantially planar upper bearing surface 116a and may include an aperture 116b therein. As described further below, the upper bearing surface 116a and the aperture 116b are employed to movably couple the talus body 100 with the core 200.

A lower portion 118 of the frame structure 110 may provide a substantially planar lower bearing surface 118a and may include a pair of apertures 118b therein. As described further below, the lower bearing surface 118a and the apertures 118b are employed to movably couple the talus body 100 with the toe 300.

The front section 120 of the talus body 100 may generally comprise a shaped vertical column. The front section 120 may include a relatively thicker (laterally) front end 122 with shaped side bearing surfaces 122a that taper away from a thicker central portion, as described in more detail below. As described further below, the bearing surfaces 122a of the front end 122 are engaged and constrained by corresponding surfaces of the core 200 during operation of the artificial foot 10.

The front section 120 of the talus body 100 may further include a reinforced aperture or bearing 124 for retaining a lateral axle 124a. In some embodiments, the lateral axle 124a may be formed integrally with the talus body 100, eliminating any need for an aperture or bearing.

One or more bumpers 130 may be mounted on a bottom surface or surfaces 132 of the frame structure 110 of the talus body 100. Such bumpers 130 may be made of urethane, for example, and may serve to cushion and/or limit downward movement of the talus body 100 during operation of the artificial foot 10. Alternatively, the bumper(s) 130 may be mounted on the core 200, as appropriate or desired.

The core 200 may include a heel-strike portion 210, a mid-foot bearing portion 220 and a toe engagement portion 230. These portions may be formed as an integral structure, for example, by injection molding a high performance plastics material, such as polyetheretherketone (PEEK), a bearing-grade plastic.

The heel strike portion 210 may extend rearwardly beneath the frame structure 110 of the talus body 100 when the artificial foot 10 is assembled, and may provide impact absorption for the artificial foot 10 in use. In embodiments, the heel strike portion 210 may include upwardly extending side walls 212 that may increase the strength and resilience of the heel strike portion 210. In embodiments, the side walls 212 may extend substantially to a rear edge 214 of the heel strike portion 210.

It should be noted that the outer surfaces of the core 200 may be suitably shaped to facilitate fitting the artificial foot 10 into shoes or other prosthetic devices. In particular, the outer dimensions of the core 200 may be symmetrical about a longitudinal axis (heel-to-toe) of the artificial foot 10. Thus, the core 200 may be a universal device, serving as either a right or left foot as desired.

The heel strike portion 210 and/or the mid-foot bearing portion 220 may include a retaining means 216, such as a recess and aperture as shown, configured to receive and retain one end of a tension/limit member, as described further below, for movably coupling the talus body 100 with the core 200.

The mid-foot bearing portion 220 may include a central channel or cavity 222. The cavity 222 may open rearwardly and upwardly, and may define side bearing surfaces 222a. The side bearing surfaces 222a may be shaped or otherwise configured to cooperate with the side bearing surfaces 122a of the front end 122 of the talus body 100 to constrain relative motion of the talus body 100 and the core during operation of the artificial foot 10.

The mid-foot bearing portion 220 may also include a pair of bearing recesses 224 defined in opposite inner walls of the central channel or cavity 222. The bearing recesses 224 may open rearwardly and may be configured to receive and retain respective bearings 226, each of which is configured to receive and retain an opposite end of the lateral axle 124a of the front section 120 of the talus body 100 when the artificial foot 10 is assembled. The bearing recesses 224 may be tapered to narrow in a forward direction, that is, toward the toe engagement portion 230. The bearings 226 may be similarly tapered to fit within the respective bearing recess 224. The lateral axle 124a may have a suitable degree of play within the bearings 226, for example, to avoid friction and heat. Further, the bearing recesses 224 may allow for a desired degree of movement of the respective bearings 226 within the bearing recesses 224. For example, a desired degree of play should exist to allow the interaction of the side bearing surfaces 122a of the front end 122 of the talus body 100 with the side bearing surfaces 222a of the cavity 222 of the mid-foot bearing portion 220 of the core 200 to govern a desired range of motion between the talus body 100 and the core 200 during operation of the artificial foot 10. When coupled, the lateral axle 124a, bearings 226 and bearing recesses 224 thus may allow constrained relative rotation and translation between the talus body 100 and the core 200.

It should be understood that, in some embodiments, the bearings 226 may be omitted and that the bearing recesses 224 may be configured to serve as bearings themselves. In such case, the bearing recesses 224 need only be configured to cooperate with the lateral axle 124a of the talus body 100 to provide the desired limited relative movement.

The mid-foot bearing portion 220 may also include a pair of generally longitudinal channels 228, each of which opens to the heel-strike portion 210 and to the toe engagement portion 230. As described further below, the channels 228 may be configured to receive respective tension/limit members to couple the talus body 100 with the toe 300.

The toe engagement portion 230 may generally comprise a narrowed section or protrusion relative to the mid-foot bearing portion 220. As described further below, the toe engagement portion 230 may include curved or rounded upper and lower front edges to accommodate relative rotation of the toe 300, to provide a weight bearing surface during use, and to provide a smooth contact surface for an upper tension/limit member of the toe 300.

An upper portion of the toe engagement portion 230 and/or the mid-foot bearing portion 220 may include a retaining means 232, such as the recess and groove shown, configured to receive and retain an end of a tension/limit member 240 configured to constrain rotation of the toe 300 relative to the core 200.

A front end face 234 of the toe engagement portion 230 may include an upper pair of recesses 236 and a lower pair of recesses 238. Each of the recesses 236, 238 may be generally spherical and open forwardly. Each of the recesses 236, 238 may further include a narrower lateral portion that opens to a respective side of the toe engagement portion 230. As such, each of the recesses 236, 238 is configured to receive and movably retain one end of a respective link 250. In particular, the links 250 may be held in the recesses 236, 238 by a suitable mechanical assembly 234c, such as a washer and fastener as illustrated.

Each of the links 250 may comprise a metal, such as stainless steel, for strength and wear resistance. Each link 250 may be a unitary member in a generally “dumbbell” configuration, with spherical end portions interconnected by a narrower, centralized cylindrical portion. It should be understood that the links 250 need not be identical, nor symmetrical, although shown as such. Some or all of the links 250 may also be cable, with spherical swages on each end.

The toe 300 may comprise a generally U-shaped rear bracket portion 310 and a tapered, forwardly extending plantar portion 320. A rear end face 312 of the bracket portion 310 may include an upper pair of recesses 314 and a lower pair of recesses 316. Each of the recesses 314, 316 may be generally hemispherical and open rearwardly. Each of the recesses 314, 316 may further include a narrower lateral portion that opens to a respective side of the toe 300.

The rear bracket portion 310 may be configured to receive the toe engagement portion 230 at least partially therein when the artificial foot is assembled. As each of the recesses 314, 316 is configured to receive and movably retain the other end of a respective link 250 that is engaged by the respective recess 236, 238 of the toe engagement portion 230, the links 250 movably couple the toe engagement portion 230 of the core 200 with the toe 300 to allow the toe 300 to rotate generally about a lateral axis during use of the artificial foot 10. Using pairs of links 250, an overrange of rotation of the toe 300 relative to the core 200 may be possible. For example, the links 250 in the upper recesses 236, 314 may remain engaged while the links 250 in the lower recesses 238, 316 partially disengage, and vice versa, as appropriate or desired, with other tension/limit members maintaining joint integrity.

The rear bracket portion 310 may include a pair of retaining means 318, such as recesses and bores as shown, on opposite sides thereof, each of which is configured to receive and retain an end of a tension/limit member 260 configured to constrain relative movement of the toe 300 and the talus body 100, as described further below.

The plantar portion 320 may also include a retaining means 322, such as a recess and bore or aperture as shown, configured to receive and retain the other end of the tension/limit member 240 configured to constrain rotation of the toe 300 relative to the core 200.

Returning to the tension/limit members 260 coupling the toe 300 with the talus body 100 when the artificial foot is assembled, each of the members 260 extends through a respective one of the longitudinal channels 228 in the mid-foot bearing portion 220 of the core 200, and through a respective one of the apertures 118b in the lower bearing surface 118a of the lower portion 118 of the frame structure 110 of the talus body 100.

An opposite end 262 of each of the tension/limit members 260 may be retained by a toe spring carrier assembly 270 disposed adjacent the lower bearing surface 118a of the lower portion 118 of the frame structure 110 of the talus body 100. The opposite ends 262 may be retained in any suitable manner, for example, by a recess and aperture formed in a closed end 272 of an encasement 274. Each of the ends of the tension/limit members 260 may be secured using a respective spherical bushing 264 (FIG. 7) to provide a bearing surface for smooth operation of the artificial foot 10.

The encasement 274 may define respective cavities 276 with an open end opposite the closed end 272, each of the cavities 276 being configured to receive and retain a respective elastomeric members 278 through which the respective tension/limit members 260 extend axially. Additionally or alternatively, the cavities 276 may receive and retain coil springs. In a relaxed, or substantially uncompressed state, the elastomeric members 278 (and/or coil springs) extend beyond the encasement 274. During use, when the toe 300 is rotated upwardly the tension/limit members 260 will be tensioned and pull encasement 274 of the toe spring carrier assembly 270 toward the lower bearing surface 118a, compressing the elastomeric members 278 (and/or coil springs). Engagement of the encasement 274 with the lower bearing surface 118a provides a stop limit through the tension/limit members 260 for the upward rotation of the toe 300. Further, because the elastomeric members 278 are compressed during such rotation of the toe 300, whether or not the stop limit is reached, the elastomeric members 278 may provide actuation for downward rotation of the toe 300 through the tension/limit members 260, for example, during toe-off of a stepping movement with the artificial foot 10. Also during use, the elastomeric members 278 (and/or coil springs) may be compressed by movement of the talus body 100, the “pre-loading” the toe 300. This may enhance stability and store additional energy to be released during toe-off.

Returning to the movable coupling of the talus body 100 with the core 200 via the upper bearing surface 116a and the aperture 116b, a tension/limit member 280 may have one end engaged by the retaining means 216 and may extend through the aperture 116b, with an opposite end 282 engaged by a core spring carrier assembly 290 disposed above the upper bearing surface 116a of the upper portion 116 of the frame structure 110 of the talus body 100. The opposite end 282 may be retained in any suitable manner, for example, by a recess and aperture formed in a closed end 292 of an encasement 294. In the embodiment shown, the tension/limit member 280 may be a solid rod, for example, made of steel or other suitable metal, which may include threaded ends for securing to the retaining means 216 and/or the encasement 294. In particular, each of the ends of the tension/limit member 280 may be secured using a respective bushing 282 (FIG. 7) to provide a bearing surface for smooth operation of the artificial foot 10.

In the embodiment illustrated, the use of a solid rod for the tension/limit member 280 may provide particular advantages. For example, fatigue strength may be improved, or at least defined, for a rod as compared to a rope for this use. Further, cost may be reduced and ease of assembly may be facilitated using a rod. For example, the rod may comprise a socket head cap screw.

The encasement 294 may define a plurality of cavities 296, each with an open end opposite the closed end 292, each of the cavities 296 being configured to receive and retain a respective coil spring 298 axially oriented generally parallel to the tension/limit member 280. Additionally or alternatively, the cavities 296 may receive and retain an elastomeric material. In a relaxed, or substantially uncompressed state, the coil springs 298 (and/or elastomeric material) extend beyond the encasement 294. During use, when the talus body 100 is rotated forwardly relative to the core 200 the tension/limit member 280 will pull encasement 294 of the spring carrier assembly 290 toward the upper bearing surface 116a, compressing the coil springs 298 (and/or elastomeric material). Engagement of the encasement 294 with the upper bearing surface 116a provides a stop limit through the tension/limit member 280 for the forward rotation of the talus body 100. Further, because the coil springs 298 are compressed during such rotation of the talus body 100, whether or not the stop limit is reached, the coil springs 298 may provide actuation for downward rotation of the core 200 through the tension/limit member 280, for example, during a forward stepping movement with the artificial foot 10.

It should be understood that the constraints provided by the various tension/limit members may not only influence motion of the joints 12 and 14, but also may stabilize the joints 12 and 14. Moreover, as appropriate or desired, the tension ropes may be configured to store and release energy during walking movements of the artificial foot 10, thereby mimicking performance characteristics of the human foot.

By employing grooves for the tension/limit members, particularly tension/limit ropes, the members may be maintained in proper place and may be less likely to be damaged. Although not specifically depicted in the drawings for the sake of simplicity, it should be understood that means for adjusting the tension of the various tension ropes may be included in a practical implementation. Any suitable adjustment mechanism may be employed.

The bottom of the core 200, and the bottom of the toe 300 to some degree, may serve as load-bearing surfaces during walking movements of the artificial foot 10. The loads may be transferred through the joints 12 and 14 to provide a more realistic walking performance of the artificial foot 10.

It should be noted that for each of the tension members secured to a respective member by a ball-end, as illustrated in some of the figures, a bushing made of a suitable material, such as a plastics material, may be included to provide a suitable surface for movement of the ball relative to the respective member. This may facilitate smooth movement of the corresponding ropes and/or prevent wear at the rope connections.

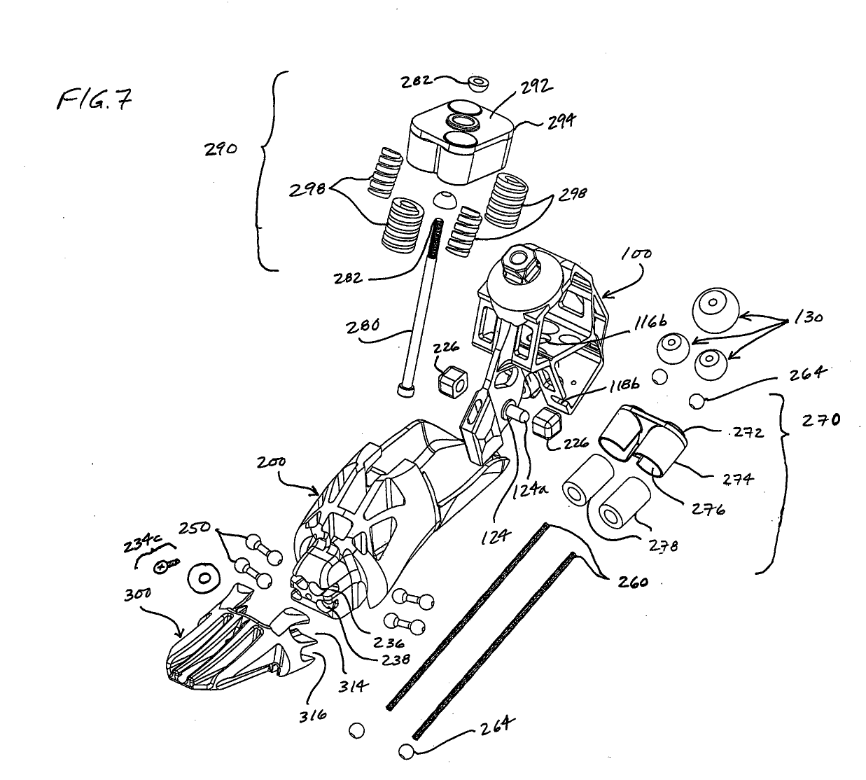

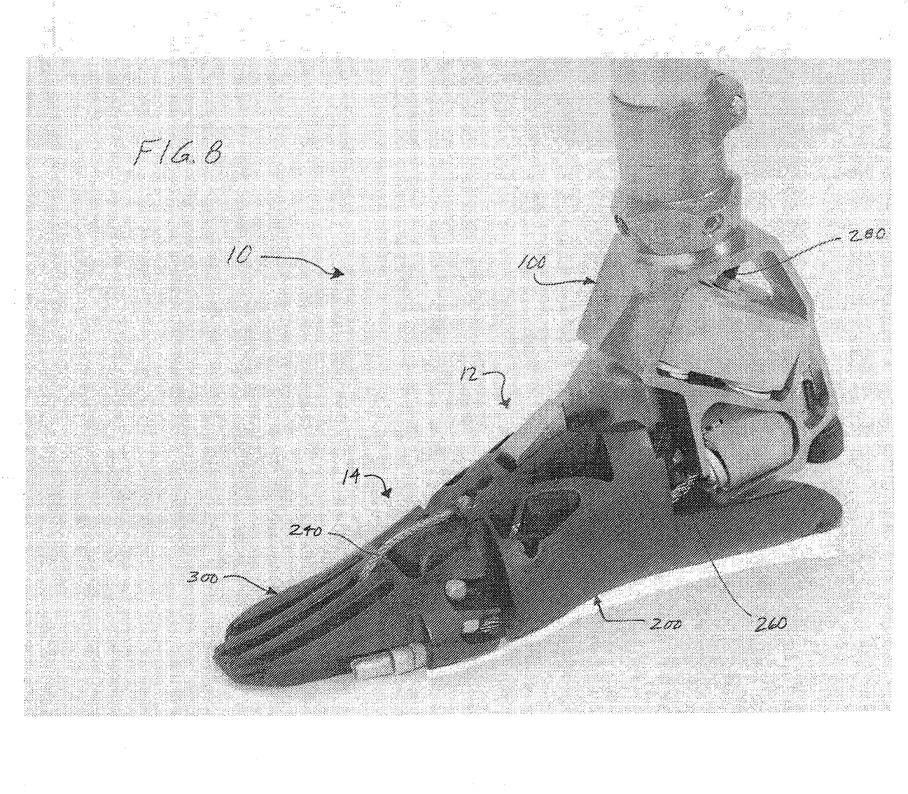

Turning to FIGS. 9-13 of the talus body 100 in isolation, various features described above are illustrated without obstruction. The frame structure 110 including the upper portion 112, the mid-portion 116, the lower portion 118 and cutouts 102 is shown. Again, the coupling or engagement means 114 may be implemented as a well-known “pyramid connector.”

The mid-portion 116 of the frame structure 110 provides the substantially planar upper bearing surface 116a and includes the aperture 116b, which are employed to movably couple the talus body 100 with the core 200 via the tension/limit member 280 and the core spring carrier assembly 290. The lower portion 118 of the frame structure 110 provides the substantially planar lower bearing surface 118a and include the apertures 118b, which are employed to movably couple the talus body 100 with the toe 300 via the tension/limit member 260 and the toe spring carrier assembly 270.

It should be noted that the relative angle of the upper bearing surface 116a may be configured such that the maximum loading of the tension/limit member coupling the talus body and the core occurs when the longitudinal axis of that tension/limit member is substantially perpendicular to the plane of the upper bearing surface 116a. This configuration may help to maintain direct compression of the coil springs (and/or elastomeric material) and limit translation of the coil springs (and/or elastomeric material) along the upper bearing surface 116a.

Similarly, it should be noted that the relative angle of the lower bearing surface 118a may be configured such that when the mid-foot joint and the toe joint are at a maximum range of motion, the plane of the lower bearing surface 118a is substantially perpendicular to the longitudinal axis of the tension/limit member or the longitudinal axis that runs from the toe. This configuration may help to maintain direct compression of the elastomeric members (and/or coil springs) and limit upward/downward movement of the elastomeric members (and/or coil springs) along the lower bearing surface 118a. Also, a bottom wall surface 118c of the lower portion 118, upon which the encasement of the toe spring carrier assembly may movably rest, may be disposed at a relatively small angle (such as 2 or 3 degrees) relative to the lower bearing surface 118a, for example, to accommodate for a corresponding degree of draft that may be employed when the encasement is made by an injection molding process.

The front section 120 including the front end 122 with shaped side bearing surfaces 122a may be understood particularly from the talus body 100 shown in isolation. As discussed herein, the bearing surfaces 122a of the front end 122 are engaged and constrained by corresponding surfaces of the core 200 during operation of the artificial foot 10. In general, the front and rear of the bearing surfaces 122a may be configured to limit rotation of the core 200 relative to the talus body 100 about a substantially vertical axis, substantially parallel to the pylon for example. The upper and lower of the bearing surfaces 122a may be configured to limit rotation (e.g., twisting) of the core 200 relative to the talus body 100 about a substantially horizontal axis, substantially in-line with the talus-core-toe assembly. The particular angles of the bearing surfaces 122a and the distances between the bearing surfaces 122a and the corresponding bearing surfaces 222a in the cavity 222 of the core 200 may be critical to the design as such parameters may control the amount of relative motion that is allowed between the talus body 100 and the core 200, in addition to the limits placed on rotation about the lateral axle 124a. The bearing surfaces may be designed to contact corresponding bearing surfaces with maximum amounts of surface area during use to decrease stress of the surfaces and increase surface longevity.

It should be noted that the opposing upper/lower bearing surfaces 122a may not be entirely parallel so as to correspond and cooperate with the respective bearing surfaces 222a of the core 200 when the core 200 is made by an injection molding process. Specifically, the upper/lower bearing surfaces 122a may be 2 to 3 degrees from parallel to account for the 2 to 3 degree draft angle of the injection molding process. This may also be true for the opposing front/rear bearing surfaces 122a.

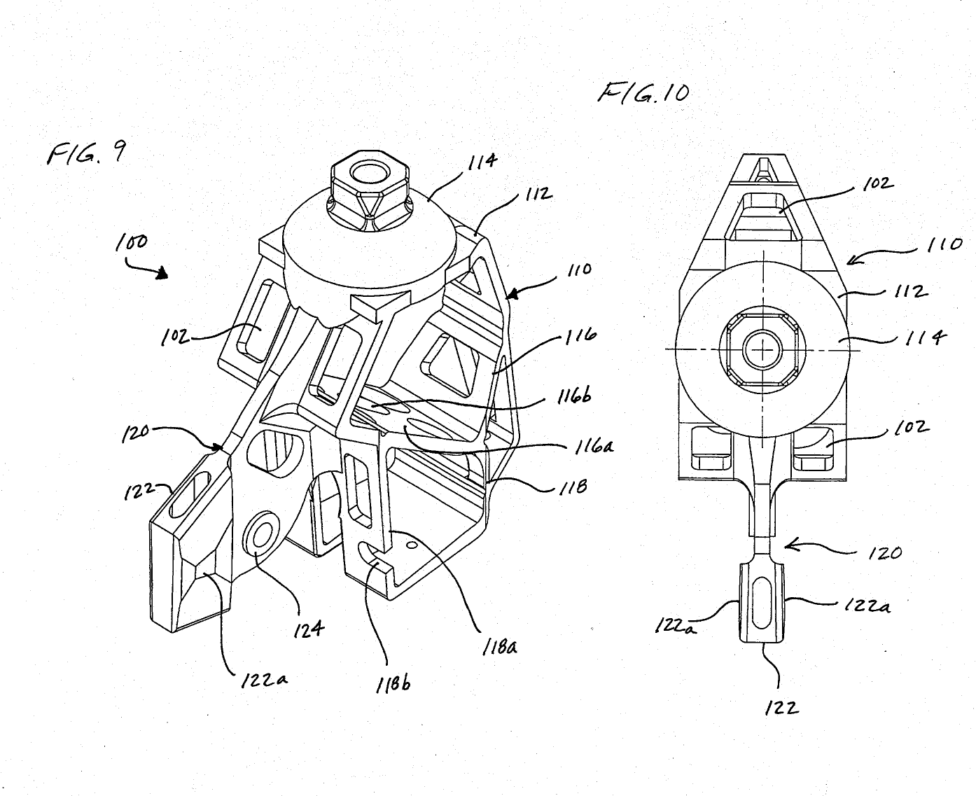

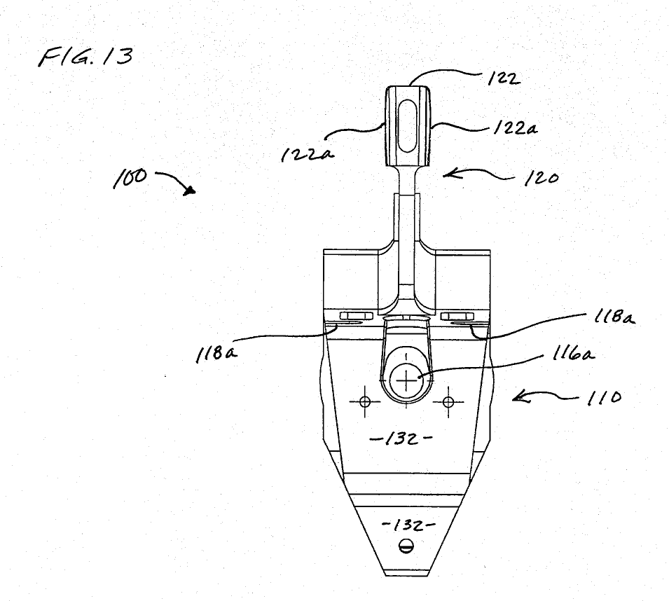

Turning to FIGS. 14-19 of the core 200 in isolation, various features described above are illustrated without obstruction. As described above, the core 200 includes the heel-strike portion 210, the mid-foot bearing portion 220 and the toe engagement portion 230. The heel strike portion 210 includes upwardly extending side walls 212 that may increase the strength and resilience of the heel strike portion 210. In embodiments, the side walls 212 may extend substantially to the rear edge 214 of the heel strike portion 210.

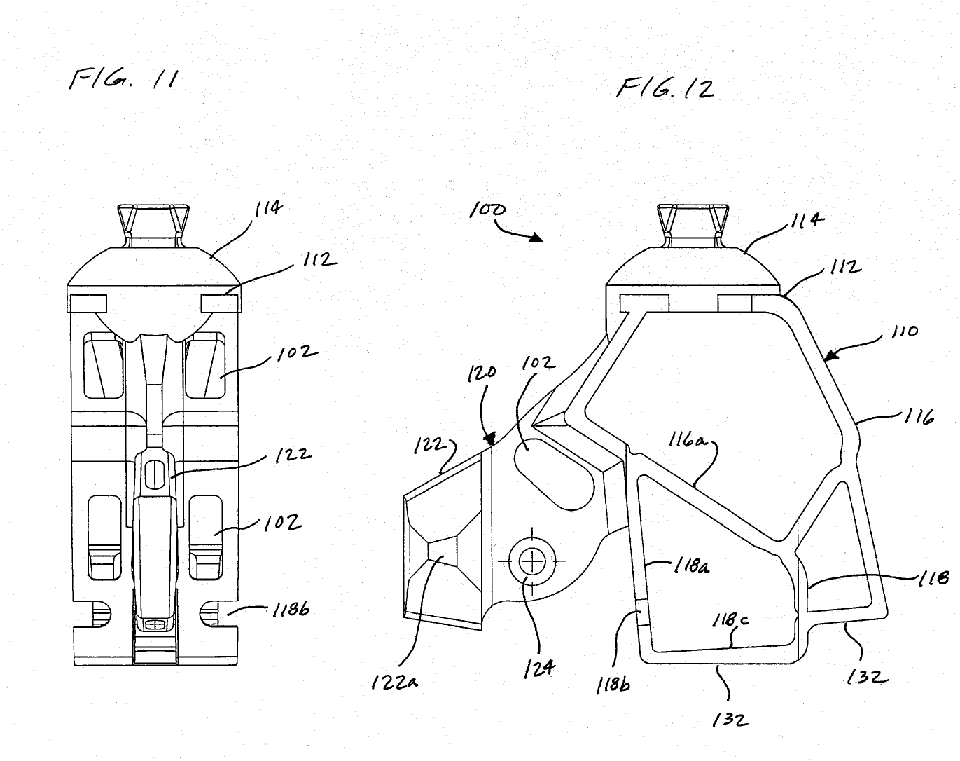

As particularly illustrated in FIGS. 16 and 17, the retaining means 216, such as the recess and aperture shown, configured for receiving and retaining one end of the tension/limit member movably coupling the core 200 with the talus body 100 opens to the bottom surface of the core 200 for ease of assembly. Also in these figures, the longitudinal channels 228 configured to receive respective tension/limit members to couple the talus body 100 with the toe 300 open to the heel-strike portion 210, on top, and to the toe engagement portion 230, on bottom.

The mid-foot bearing portion 220 includes the readwardly opening cavity 222 with side bearing surfaces 222a. The mid-foot bearing portion 220 also includes the rearwardly opening bearing recesses 224 in opposite inner walls of the cavity 222 for receiving and movably retaining the respective bearings 226 when the artificial foot 10 is assembled. As discussed above, the bearing recesses 224 may be tapered to narrow in the forward direction, and may allow for a desired degree of movement of the respective bearings 226 within the bearing recesses 224. In the embodiment shown in these figures, there is only enough room for the axle to be received, not the bearings. In this embodiment, the core may be made of a suitable bearing material, so that additional bearings may not be required.

In the embodiment shown, the toe engagement portion 230 includes a curved or rounded upper front edge 234a and a curved or rounded lower front edge 234b. In particular, the upper and lower front edges 234a, 234b may accommodate relative rotation of the toe 300 about the front end face 234 of the core 200. The rounded lower front edge 234b may provide a weight bearing surface during use, allowing a “rolling contact” surface during heel lift-off. The rounded upper front edge 234a may provide a smooth contact surface for the upper tension/limit member 240 of the toe 300.

As described above, the upper portion of the toe engagement portion 230 and/or the mid-foot bearing portion 220 may include the retaining means 232, such as the recess and groove shown, configured to receive and retain an end of a tension/limit member 240 configured to constrain rotation of the toe 300 relative to the core 200.

Further, the front end face 234 of the toe engagement portion 230 includes the recesses 236, 238 configured to receive and movably retain one end of respective links 250. In particular, the links 250 may be held in the recesses 236, 238 by a suitable mechanical assembly (not shown) or by use of adhesives.

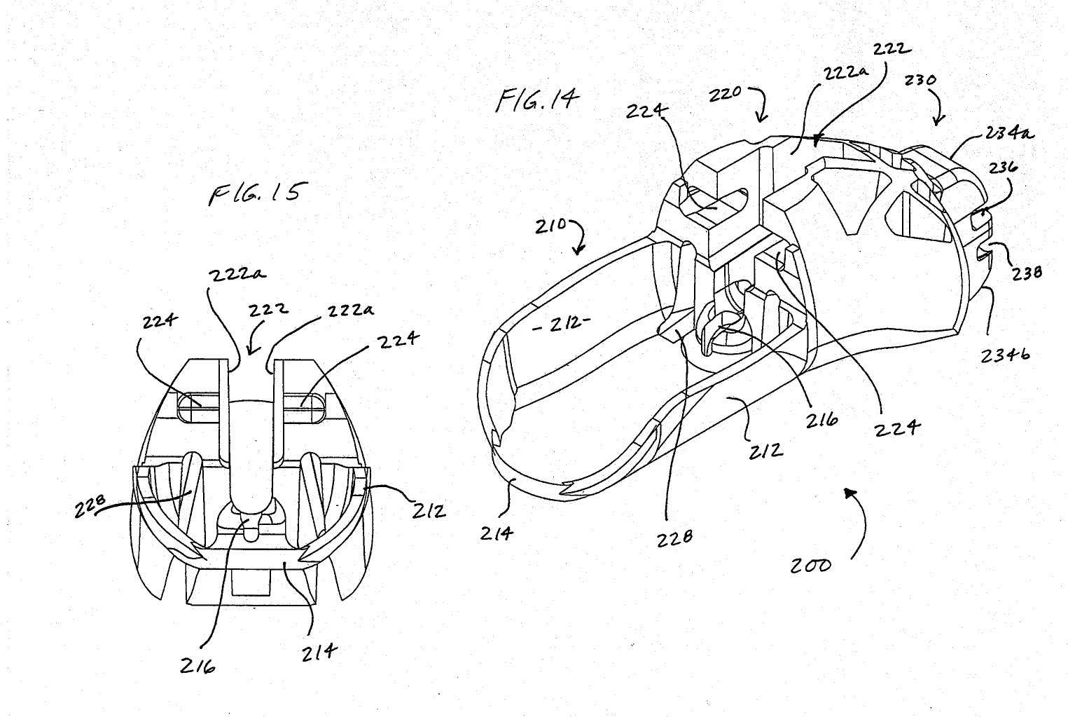

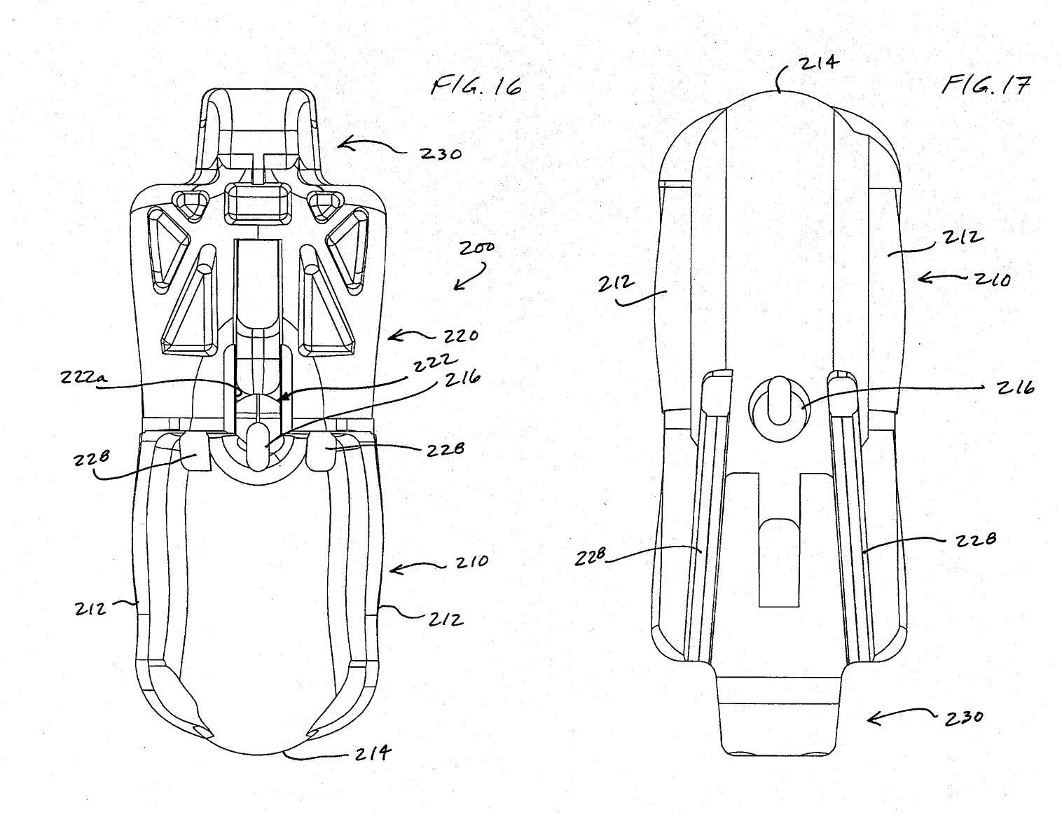

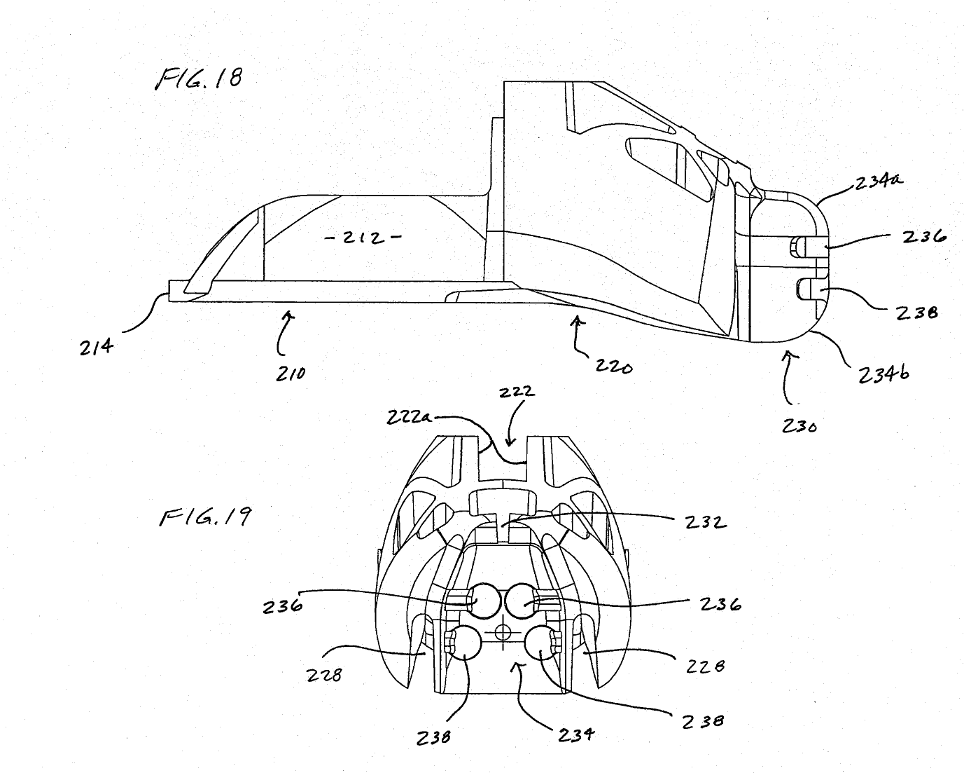

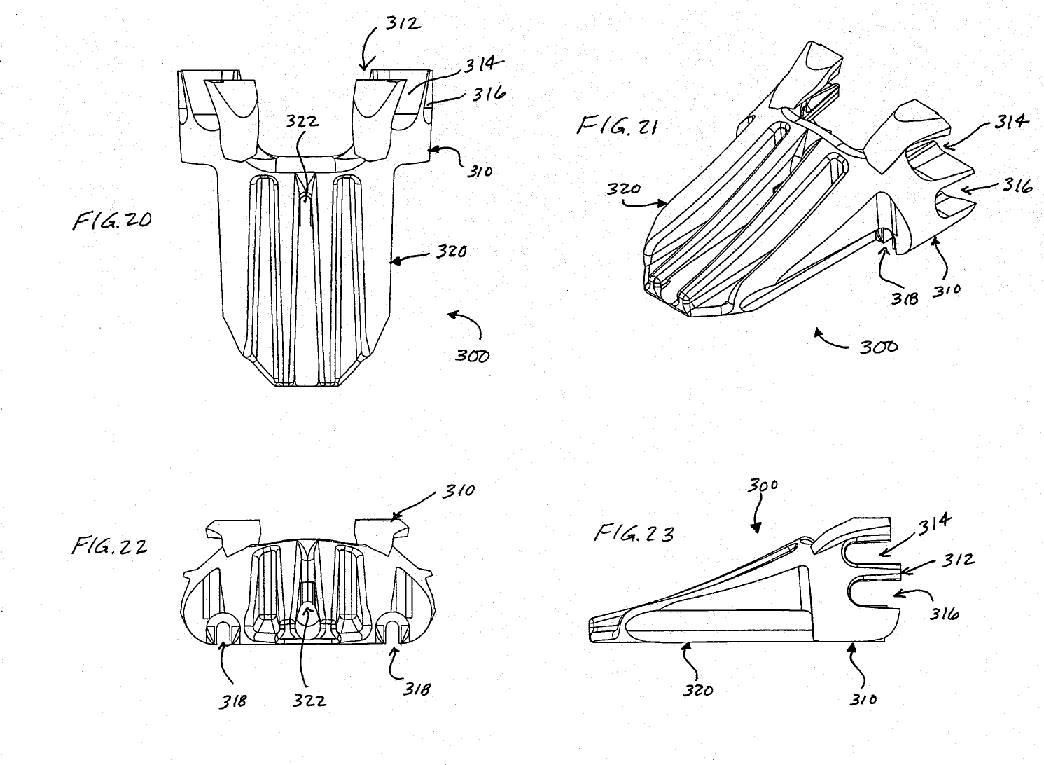

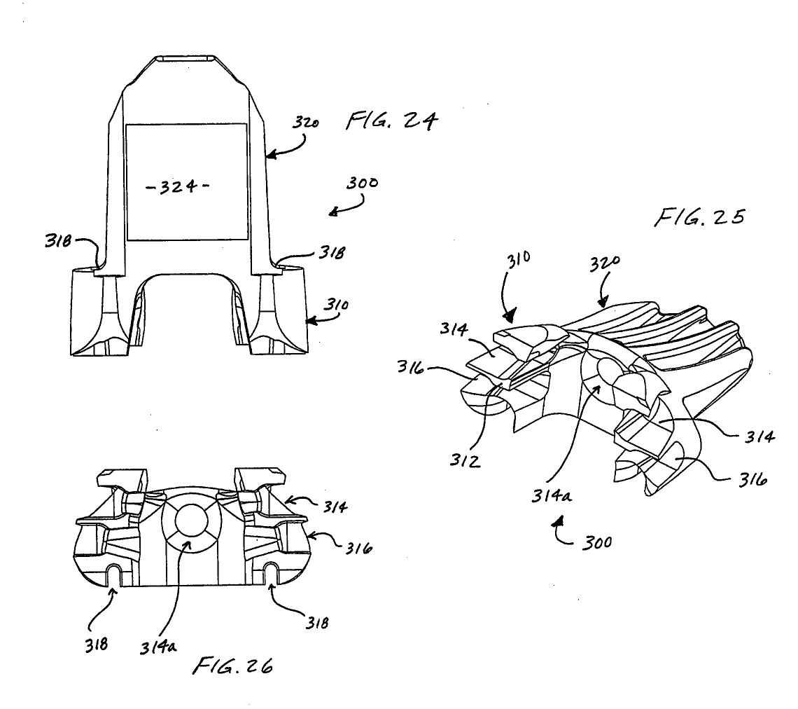

Turning to FIGS. 20-26 of the toe 300 in isolation, various features described above are illustrated without obstruction. As described above, the toe 300 includes the rear bracket portion 310 and the forwardly extending plantar portion 320. The rear end face 312 of the bracket portion 310 includes the upper recesses 314 and the lower recesses 316 for receiving and movably retaining the other end of a respective link 250 that is engaged by the respective recess 236, 238 of the toe engagement portion 230 when the artificial foot 10 is assembled, with the links 250 movably coupling the toe engagement portion 230 of the core 200 with the toe 300 to allow the toe 300 to rotate generally about a lateral axis during use of the artificial foot 10.

The rear bracket portion 310 also includes the retaining means 318 configured to receive and retain ends of the respective tension/limit members 260. Also, the rear bracket portion 310 includes a recess or relief 314a for accommodating the mechanical assembly used to hold the links 250 in the recesses 236, 238 on the front end face 234 of the toe engagement portion 230.

The plantar portion 320 includes the retaining means 322 configured to receive and retain the other end of the tension/limit member 240 configured to constrain rotation of the toe 300 relative to the core 200. As particularly shown in FIG. 22, for example, the retaining means may comprise an open cavity in an upper portion of the plantar portion 320 that opens upwardly through a relatively narrower open channel. Thus, the tension/limit member 240 may extend through the channel with its end secured in the cavity of the retaining means 322. It should be noted that the retaining means 322 does not extend through to a bottom plantar surface 324, to avoid any potential

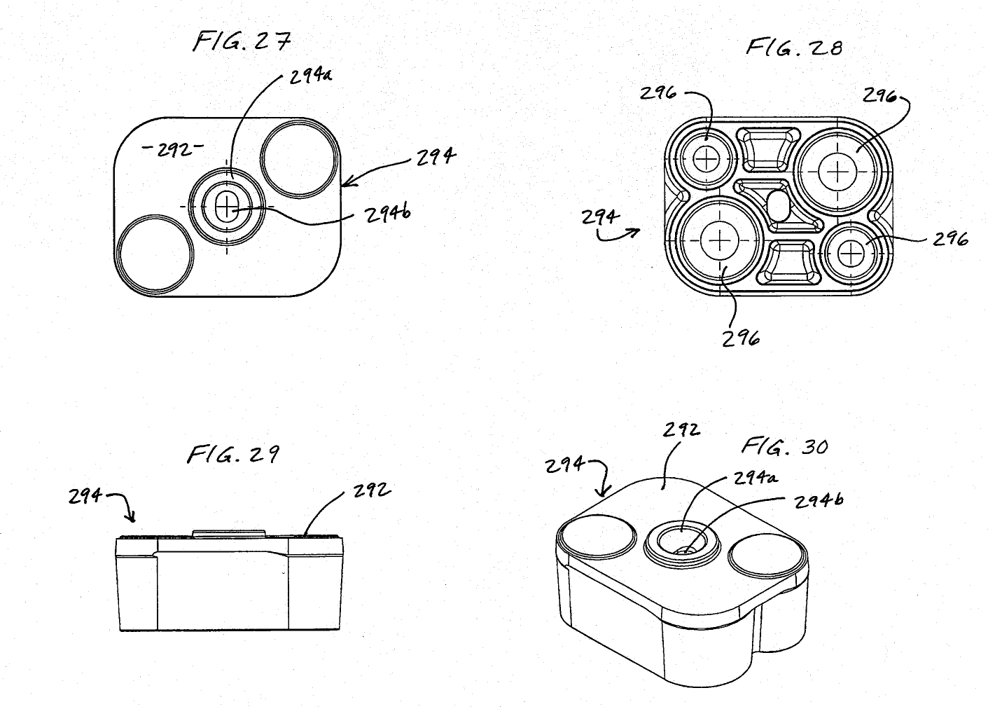

Turning to FIGS. 27-30 of the encasement 294 of the core spring carrier assembly 290 in isolation, various features described above are illustrated without obstruction. As described above, the tension/limit member 280 may have one end engaged by the retaining means 216 and may extend through the aperture 116b of the frame structure 110, with an opposite end 282 engaged/retained in a suitable manner, such as by a recess 294a and aperture 294b formed in the closed end 292 of the encasement 294.

As illustrated, the encasement 294 defines a plurality of cavities 296, each with an open end opposite the closed end 292, each of the cavities 296 being configured to receive and retain a respective coil spring, elastomeric member and/or elastomeric material, as described above. It should be understood that the cavities 296 may be of the same size, either depth and/or width, to accommodate corresponding elements as desired.

Further, it should be understood that the cavities 296 may be of different sizes, either depth and/or width, to accommodate differing corresponding elements as desired. It may be desirable to have one or more elements loaded in succession (variable timing of loading/actuation) by having the elements extend different lengths from the encasement 294. This may be achieved by varying the lengths of the elements, the depths of the cavities, or both. Similarly, different loading characteristics may be achieved by varying the length and/or width of the elements and the corresponding cavity dimensions.

For example, the cavities 296 may be configured to hold the elements at different depths such that not all elements begin to be compressed by the motion of the mid-foot joint. This action directly impacts the torque response curve of the mid-foot joint. If the larger elements (e.g., springs) contact the bearing surface first, there is a larger initial stiffness of the joint. If the smaller elements contact the bearing surface first, there is a smaller initial stiffness of the joint. Thus, the length of the elements may be used to “tune” the torque response curve.

In the embodiment shown, even if different sized cavities 296 are employed, the cavities 296 may be disposed symmetrically relative to the longitudinal (heel-to-toe) axis and/or the lateral axis of the artificial foot 10 to provide a neutral response.

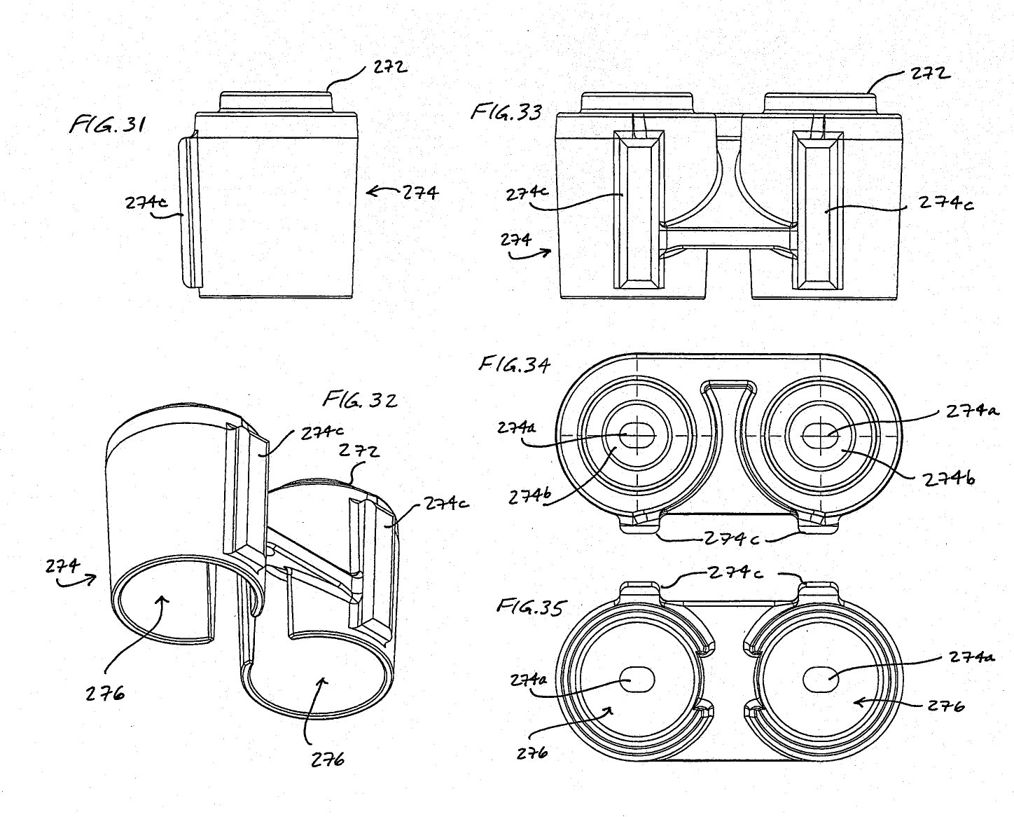

Turning to FIGS. 31-35 of the encasement 274 of the toe spring carrier assembly 270 in isolation, various features described above are illustrated without obstruction. As described above, each of the tension/limit members 260 may have one end engaged by the respective retaining means 232 and may extend through the respective aperture 118b of the frame structure 110, with an opposite end 262 engaged/retained in a suitable manner, such as by a recess 274a and aperture 274b formed in the closed end 272 of the encasement 274.

As illustrated, the encasement 274 defines a plurality of cavities 276, each with an open end opposite the closed end 272, each of the cavities 276 being configured to receive and retain a respective coil spring, elastomeric member and/or elastomeric material, as described above. It should be understood that the cavities 276 may be of the same size, either depth and/or width, to accommodate corresponding elements as desired. Further, it should be understood that the cavities 276 may be of different sizes, either depth and/or width, to accommodate differing corresponding elements as desired.

As discussed above, it may be desirable to have one or more elements loaded in succession (variable timing of loading/actuation) by having the elements extend different lengths from the encasement 274. In the embodiment shown, varying the relative performance of the elements disposed in the cavities 276 will vary the relative performance of the right and left tension/limit members 260, thus providing a non-linear/off-axis bias to the operation of the artificial foot 10. In other words, differences in the cavities 276 and/or differences in the elements (springs, elastomeric members) may provide a biased response, for example, to define a right or left prosthetic foot, or for other reasons. Such left/right differences may also be achieved by different properties of the respective tension/limit members 260, such as having different relative lengths, so that one side of the toe 300 has a greater range of motion than the other side.

The encasement 274 may further include raised surfaces, projections or rails 274c as illustrated. The rails 274c may increase wear resistance of the encasement 274 for its sliding movement against the bottom wall surface 118c. The rails 274c may also accommodate a 2 to 3 degree draft angle that the encasement 274 may have when made by an injection molding process. Further, the rails 274c may be set back from the open end of the encasement to avoid any potential interference with a filet or rounded corner in the frame structure 110 between the bearing surface 118a and the bottom wall surface 118c.

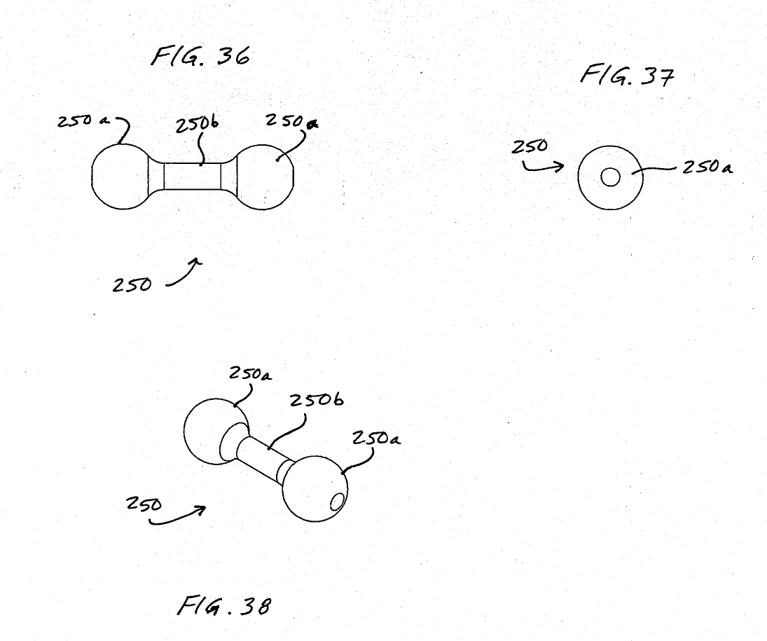

Turning to FIGS. 36-38 of the link 250 configured to movably couple the core 200 and the toe 300, various features described above are illustrated without obstruction. As described above, each link 250 may be a unitary member in a generally “dumbbell” configuration. The link 250 may have spherical end portions 250a interconnected by a narrower, centralized cylindrical portion 250b. It should be understood that other configurations of the links 250 are possible as well, although the curved surfaces of the “dumbbell” configuration may provide less potential for fatigue and/or failure of the parts of the artificial foot 10 contacted by the links 250.

Operation of the artificial foot 10 during walking movement may be described as follows. Beginning with heel strike, the heel strike portion 210 of the core 200 may absorb impact. The talus body 100 may move downward toward the heel strike portion 210 with the bumpers 130 (when provided) contacting the upper surface of the heel strike portion 210 to absorb the heel strike. The talus body 100 may rotate (counter-clockwise in FIG. 2) relative to the core 200 during downward movement of the talus body 100, moving the bearings 226 to a top of the bearing recesses 224 of the talus body core 200.

The talus body 100 may then rotate about a virtual lateral axis in an opposite direction (clockwise in FIG. 2) relative to the core 200 until the artificial foot reaches a neutral (standing support) position. In the neutral position, the bearings 226 may return to a bottom of the bearing recesses 224. Further in this position, the artificial foot may be supported via the bottom of the core 200 as a contact/support/load-bearing surface.

From the neutral position, tibial progression in a forward direction causes the talus body 100 to rotate about the axle 124a of the first joint 12, via the bearings 224 when present. The coil springs 298 in the spring carrier assembly 290 are loaded against the upper bearing surface 116a and the coil springs 278 in the spring carrier assembly 270 are loaded against the lower bearing surface 118a as the tension/limit member 260 is loaded.

Continued tibial progression forward causes heel rise or lift-off as well as rotation of the toe 300 relative to the core 200. As the heel rises, the artificial foot 10 is supported by the toe engagement portion 230 of the core 200, with the toe 300 continuing to rotate. Such movement may occur until the limits of the tension/limit members 240, 260, 280 are reached.

In this embodiment, the tension/limit members 240 and 260 may have some elastic properties. Thus, the tension/limit members 240, 260 may be stretched by the rotation of the toe 300. One example of an elastic material for the tension/limit members 240, 260 is Nitinol. In such case, the stretching will be a maximum at the phase change for Nitinol. Once the limit(s) of the tension/limit member(s) 240, 260 has/have been reached, the artificial foot 10 may push off with the toe 300. In particular, the toe 300 may push off in a forward and upward direction, releasing energy stored in the tension/limit members 240, 260 in that direction as the artificial foot 10 is lifted by the user, in addition to the energy stored by the coil springs 278, 298 discussed above.

In operation, embodiments may provide improved movements for an artificial foot device. For example, if the foot encounters an uneven surface, such as a relatively small rock, and a portion of the artificial foot is placed at an angle about its longitudinal axis by such, the limited movement provided in one or both of the joints about the longitudinal axis may allow compliance to avoid translating at least part of the angle to the attachment pylon and/or the user's leg and/or body. In particular, if the uneven surface occurs under the toe and/or the core-assembly, the mid-foot joint may permit constrained movement to accommodate the resulting angle. If the uneven surface occurs under the toe, the toe joint may permit constrained movement to accommodate the resulting angle. In either case the other of the two joints may also contribute to accommodation of the resulting angle.

Also, the limited movement provided in one or both of the joints about a substantially vertical axis (e.g., substantially perpendicular to the longitudinal axis) may allow compliance to allow a user to change direction of travel while ambulating. Although both joints may contribute to the compliance, in some embodiments, the toe joint may provide a primary or sole contribution to the compliance. In some embodiments, the compliance may be provided without substantial energy storage, for example, to avoid undesirable backlash from the change in direction. Similarly, in some embodiments, the mid-foot joint may provide a primary or sole contribution to the compliance.

It should be understood that the various tension members may be of varying degree of flexibility and/or elasticity. Various materials may be employed for the tension members, as well as for the discontinuous members of the artificial foot. The materials may be sufficiently rigid, strong, flexible, as appropriate for the function of the particular member. In some aspects, flexibility of a particular tension member the may not be important, as long as the design maintains the member loaded primarily in tension. The weight of the user and the selected use by that user may be considered when selecting materials. For example, stronger materials may be required when the user intends to jump and land hard as compared to when the user merely intends to walk. Useful materials for discontinuous members may include metals and/or plastics. Useful materials for tension members may include steel wire rope and aramid fiber ropes. Metal, ceramic and/or plastic bearings may be also useful in the practice of the invention. Preferably, the members of the joints and foot parts of the invention may be made from aluminum (e.g., 7075 T6), steel wire rope, tool steel, plastics and/or other suitable materials based on desired rigidity, flexibility, strength, toughness and the like.

While certain exemplary embodiments have been described above in detail and shown in the accompanying drawings, it is to be understood that such embodiments are merely illustrative of and not restrictive of the broad invention. In particular, it should be recognized that the teachings provided herein apply to a wide variety of systems and processes. It will thus be recognized that various modifications may be made to the illustrated and other embodiments described herein, without departing from the broad inventive scope thereof. In view of the above it will be understood that the invention is not limited to the particular embodiments or arrangements disclosed, but is rather intended to cover any changes, adaptations or modifications which are within the scope and spirit of the disclosure provided herein.

1. (canceled)

2. An artificial foot device, comprising:
a talus body;
a core pivotally coupled to talus body at a first joint;
a toe pivotally coupled to the core at a second joint; and
a resilient member assembly operably coupled between the toe and talus body,
wherein, when the toe is caused to pivot about the second joint, the resilient member assembly provides a torque response for the toe and a range of motion limit for the toe.

3. The foot device of claim 2, further comprising a tension member comprising a first end coupled to the toe, a second end operably coupled to the talus body, and a length between the first end and second end that extends along a portion of the core.

4. The foot device of claim 3, wherein the tension member displaces along the portion of the core when the toe is caused to pivot at the second joint.

5. The foot device of claim 3, wherein the tension member comprises a rope-like member.

6. The foot device of claim 3, wherein the resilient member assembly is operably coupled to the first tension member.

7. The foot device of claim 6, wherein the resilient member assembly is coupled between the second end and the talus body.

8. The foot device of claim 7, wherein the resilient member assembly comprises at least one of a spring or elastomeric member.

9. The foot device of claim 8, wherein the resilient member assembly further comprises a rigid body, the rigid body providing the range of motion limit for the toe and the at least one of a spring or elastomeric member providing the torque response for the toe.

10. The foot device of claim 2, wherein the at least one of the first or second joint comprises multiple degrees of freedom.

11. The foot device of claim 2, further comprising a resilient member located between a heel portion of the core and a heel portion of the talus body, the resilient member being acted upon by both heel portions when the heel portions are brought together.

12. An artificial foot device, comprising:
a talus body;
a core pivotally coupled to talus body at a first joint;
a toe pivotally coupled to the core at a second joint; and
a resilient member assembly operably coupled between the core and talus body,
wherein, when the talus body is caused to pivot about the first joint, the resilient member assembly provides a torque response for the talus body and a range of motion limit for the talus body.

13. The foot device of claim 12, further comprising a tension member comprising a first end coupled to the core and a second end operably coupled to the talus body.

14. The foot device of claim 13, wherein the tension member comprises a rod-like member.

15. The foot device of claim 13, wherein the resilient member assembly is operably coupled to the tension member.

16. The foot device of claim 15, wherein the resilient member assembly is coupled between the second end and the talus body.

17. The foot device of claim 16, wherein the resilient member assembly comprises at least one of a spring or elastomeric member.

18. The foot device of claim 17, wherein the resilient member assembly further comprises a rigid body, the rigid body providing the range of motion limit for the talus body and the at least one of a spring or elastomeric member providing the torque response for the talus body.

19. The foot device of claim 12, wherein the at least one of the first or second joint comprises multiple degrees of freedom.

20. The foot device of claim 12, further comprising a resilient member located between a heel portion of the core and a heel portion of the talus body, the resilient member being acted upon by both heel portions when the heel portions are brought together.

21. An artificial foot device, comprising:
a toe pivotally coupled to a core at a joint; and
a link member extending across the joint and comprising a first end coupled to the toe and a second end coupled to the core,
wherein, as the toe pivots at the joint, at least one of the first end or second end momentarily reduces respective engagement with at least one of the toe or core.

22. The foot device of claim 21, wherein the link member terminates in a spherical element at the first end.

23. The foot device of claim 21, wherein the link member comprises at least a pair of link members on a first lateral side of the device and at least a pair of link members on a second lateral side of the device.

24. The foot device of claim 23, wherein each at least a pair of link members is arranged to have an upper link member and a lower link member.

25. The foot device of claim 24, wherein the lower link member momentarily reduces engagement as the upper link member momentarily increases engagement.

26. The foot device of claim 21, further comprising a tension member extending between the core and toe, the tension member limiting downward rotation of a tip of the toe.

27. The foot device of claim 26, wherein the tension member is a rope-like member.

28. An artificial foot device, comprising:
a talus body comprising a front portion comprising a first lateral side and a second lateral side, each lateral side comprising a substantially vertically extending bearing surface;
a core pivotally coupled to talus body at a first joint and comprising a slot in which the front portion is received, the slot comprising a third lateral side and a fourth lateral side, each lateral side comprising a substantially vertically extending bearing surface; and
a toe pivotally coupled to the core at a second joint;
wherein, the respective substantially vertically extending bearing surfaces of the first and second lateral sides are arranged with the respective substantially vertically extending bearing surfaces of the third and fourth lateral sides so as to allow the talus to pivot relative to the core at the first joint and while having limited lateral movement of the front portion within the gap.

29. The foot device of claim 28, wherein the limited lateral movement of the front portion within the gap comprises lateral translation.

30. The foot device of claim 28, wherein the limited lateral movement of the front portion within the gap comprises pivoting laterally in a generally horizontal plane.

31. The foot device of claim 28, wherein the limited lateral movement of the front portion within the gap comprises pivoting laterally in a generally vertical plane.

32. The foot device of claim 28, wherein the bearing surfaces of the first and second lateral sides are located forward of a pivot point of the first joint.
